(19) United States
(12) Patent Application Publication
(10) Pub. No.: US 2015/0238696 A1
(43) Pub. Date: Aug. 27, 2015

(54) METHOD OF DISPENSING MATERIAL USING A NEEDLE-LESS TIP DEVICE AND NEEDLE-LESS TIP DEVICE

(71) Applicant: David Mundschenk, Dania Beach, FL (US)

(72) Inventor: David Mundschenk, Dania Beach, FL (US)

(21) Appl. No.: 14/518,778

(22) Filed: Oct. 20, 2014

Related U.S. Application Data
(60) Provisional application No. 61/892,694, filed on Oct. 18, 2013.

(51) Int. Cl. A61M 5/30; A61M 5/315

(52) U.S. Cl. CPC A61M 5/30; A61M 5/31533; A61M 5/31583; A61M 5/3155

(57) ABSTRACT

A device and method of delivery of dispensing materials, such as liquids, solutions, gels of drugs, vitamins, herbal products, electrolytes or sugar solutions, or other health related items, to mammals, such as humans or other non-human animals is described. The delivery device provides a mechanism to deliver material to the mammal without injecting the material into the mammal. In one embodiment, a device, system or method for administering a material to an individual comprises a dispensing device having an elongated body sized and shaped to hold a vial containing a material to be distributed and stored within a vial. A dispensing tip cap is secured to one end of the dispensing device. The device, system or method may further contain a container comprising a dispensable material and may be configured contain a dose setting mechanism configured to dispense a predetermined amount of material to be dispensed.

### PRIORITY CLAIM

In accordance with 37 C.F.R. 1.76, a claim of priority is included in an Application Data Sheet filed concurrently herewith. Accordingly, the present invention claims priority to U.S. Provisional Patent Application No. 61/892,694, entitled “METHOD OF DISPENSING MATERIAL USING A NEEDLE-LESS TIP DEVICE AND NEEDLE-LESS TIP DEVICE”, filed Oct. 18, 2013. The contents of the above referenced application are incorporated herein by reference in its entirety.

### FIELD OF THE INVENTION

The present invention relates to the medical field, to a medical device, and method of use, adapted for delivery of a medical solution to a human or other non-human animal using a needleless delivery component; more particularly to a medical device, and method of use, adapted for delivery of predetermined doses of a material to an individual human or animal using a needleless delivery component.

### BACKGROUND OF THE INVENTION

Insulin pens provide an easy means to carry and inject insulin. Such devices, however, require a needle as insulin is an injectable deliverable. U.S. Pat. No. 5,104,380 describes a typical insulin pen. The reference is described as a generally pen-like syringe which incorporates a dose metering device provided by a cap. The cap is described as being rotatable with respect to a pen body to a position related to the dose of medicament, i.e. insulin, to be injected. The rotation compresses a coil spring which is prevented from unwinding by cooperating ratchet teeth. When the dose is to be injected, a trigger slide is moved to the left causing the ratchet teeth to come out of engagement. The spring is permitted to unwind, thereby rotating a drive sleeve, drive gear and a drive plunger. The drive plunger is formed with a quick pitch screw thread so that its rotational movement is accompanied by axial movement to cause medicament to be discharged from a cartridge and injected through a needle. A problem associated with this, along with other insulin pens, is the device requires the use of a needle. However, some situations require a delivery system which do not require injection of a medicament. As such, devices that use a needle when a needle is not required are cumbersome and can be dangerous.

Therefore, what is needed in the art is an improved device that provides a safer and more effective manner of administrating controlled, pre-determined doses of drugs, vitamins, solutions, and other materials using a needless system.

### SUMMARY OF THE INVENTION

The present invention relates to a device and method of delivery of dispensing materials, such as liquids, solutions, gels of drugs, vitamins, herbal products, electrolytes or sugar solutions, or other health related items, to mammals, such as humans or other non-human animals. The device improves upon existing delivery devices known in the art and allows automatic or controlled delivery of predetermined units of a particular material to the human or animal without the use of a needle. The delivery device provides a mechanism to deliver material to the mammal without injecting the material into the mammal.

In one embodiment, the present invention may be directed towards a device, system or method, for administering a material to an individual comprising a dispensing device having a first end, a second end, and an elongated body. The elongated body is sized and shaped to hold a vial containing a material to be distributed and stored within a vial. The device, system or method further comprises or uses a mechanism adapted to dispense material within said vial to a mammal. A dispensing tip cap is secured to one end of the dispensing device. The device, system or method may further contain a vial or container comprising a dispensable material and may include a dose setting mechanism configured to dispense a predetermined amount of material to be dispensed. The device or system therefore allows an individual to easily and independently dispense materials without the use of a needle.

Accordingly, it is a primary objective of the present invention to teach a dispensing device for dispensing materials, fluids, or solutions.

It is a further objective of the present invention to teach a system for dispensing materials, fluids, or solutions.

It is yet another objective of the present invention to teach a method for dispensing materials, fluids, or solutions.

It is a still further objective of the invention to teach a dispensing device for dispensing materials, fluids, or solutions utilizing a new applicator tip to deliver materials, fluids, or solutions using a needleless delivery system.

It is a further objective of the present invention to teach a dispensing device for dispensing materials, fluids, or solutions utilizing a new applicator tip to deliver materials, fluids, or solutions which provide a safer and more effective manner of administrating controlled doses of drugs, vitamins, solutions and other materials.

It is yet another objective of the present invention to teach a device or system for dispensing materials, fluids, or solutions providing a mechanism or assembly to provide dose metering of the dispensing materials, fluids, or solutions.

It is a still further objective of the invention to teach a device or system for administering doses of drugs, vitamins, solutions and other materials to various anatomical structures or body orifices such as nostrils, mouth, ear, anus, vagina, or urinary opening.

It is a further objective of the present invention to teach a device or system for administering doses of drugs, vitamins, solutions and other materials to various anatomical structures or orifices without the use of a needle.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE SEVERAL VIEWS OF THE DRAWINGS

FIG. 1 is perspective view of an illustrative embodiment of a dispensing pen in accordance with the present invention;

FIG. 2 is a exploded view of the dispensing pen shown in FIG. 1;

FIG. 3A is a front perspective view of an illustrative embodiment of a dispensing tip cap;

FIG. 3B is a back perspective view of the dispensing tip cap illustrated in FIG. 3A;

FIG. 4 is perspective view of dispensing pen in accordance with the present invention adapted to provide a mechanism for dose metering;

FIG. 5 is an exploded view of the dispensing pen capable of dose metering illustrated in FIG. 4;

FIG. 6 is a perspective view of the dispensing pen having dose metered capability to dispense the material in a predetermined amount;

FIG. 7 is a cross-sectional view of an embodiment of the first, upper elongated body component illustrating components of an illustrative example of a dose setting dispensing mechanism;

FIG. 8 is a sectional elevation of an embodiment of the dose setting dispensing mechanism.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

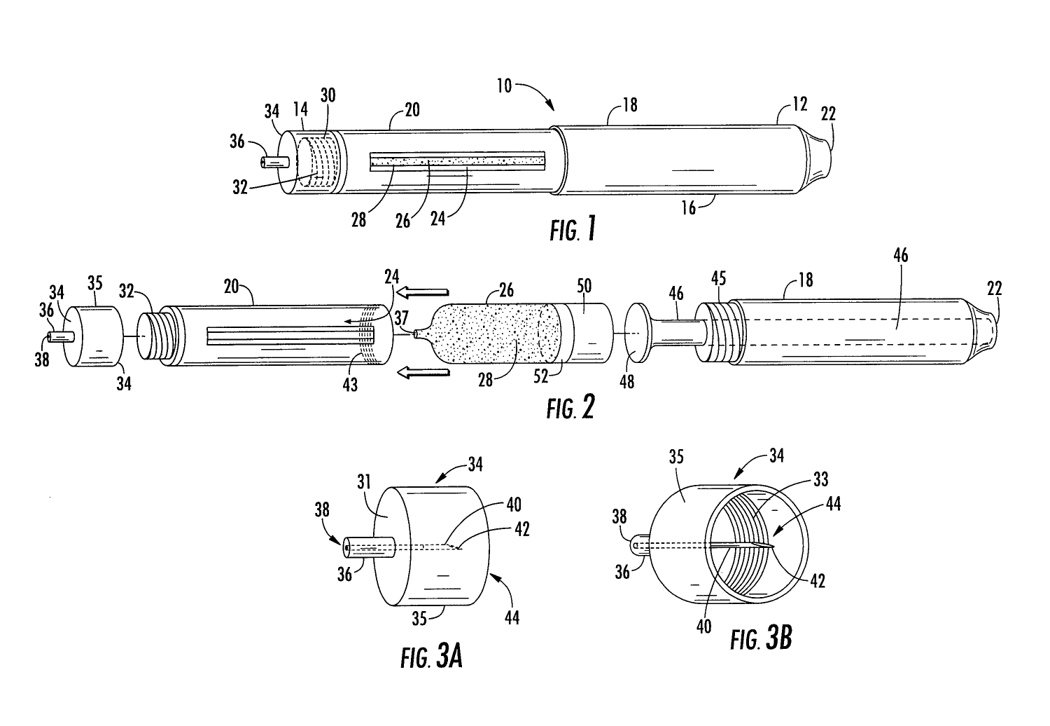

Referring to FIG. 1, an illustrative example of a device for delivering a material, referred to generally as a dispensing pen 10, is shown. The present invention will be described for use with humans, however, such use of humans is illustrative only, and the dispensing pen 10 may be used with non-human mammals or other animals. The dispensing pen comprises a first end 12, a second opposing end 14, and an elongated body 16. The elongated body 16 is shown having a generally tubular shape. The elongated body is composed of two components, a first, upper elongated body component 18 and a second, lower elongated body component 20. The upper elongated body component 18 is secured to the lower elongated body component 20 via securing mechanisms known to one of skill in the art, such as but not limited to male and female threading or via frictional fits or snap fitting.

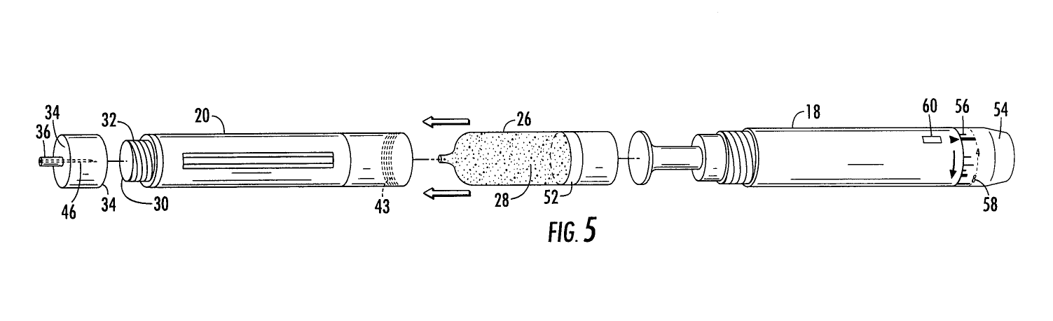

The upper elongated body component 18 is sized and shaped to house an actuating mechanism which allows for dispensing a material out of the second, opposing end 14. The material may be for example, a liquid drug, such as insulin, or vitamins, electrolytes, amino acids, herbal products, antibodies solutions, water or other hydrating solutions, and other materials or substance for which an human may want to use for health benefits. The materials dispensed are substances need by an individual, but need not be obtained through injection. The lower elongated body component 20 comprises an interior 24 and is sized and shaped to hold a vial or cartridge 26. The vial or cartridge 26 contains the material or solution 28 to be dispensed to the individual. The lower elongated body component contains a cylindrical portion 30 extending outwardly and having threading 32. The threading 32 accepts threading 33 (see FIG. 3B) of a dispensing tip cap 34, thereby tightly securing the dispensing tip cap 34 to the lower elongated body component 20.

The dispensing tip cap 34 contains an external surface 31 an a continuous side wall 35 extending away from the external surface 31, and terminating in an open end. A fluid dispensing member 36, illustrated as a non-sharp or blunted member extends outwardly away from the external surface 31. The fluid dispensing member 36 is designed to allow movement of the material or solution 28 stored within the vial or cartridge 26 to the individual via opening 38, see also FIG. 3A. While opening 38 allows for the material or solution 28 to move through the external surface 31, the fluid dispensing member 36 does not extend therethrough.

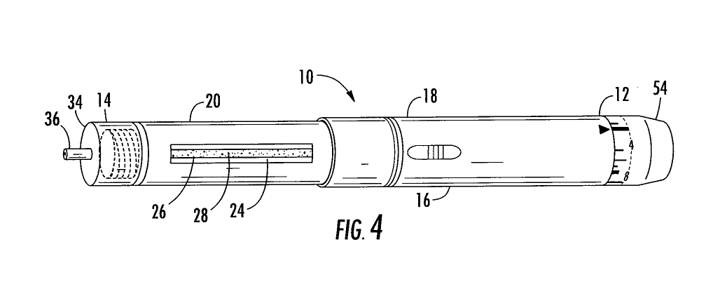

On the opposite side of the external surface 31 compared to the location of the fluid dispensing member 36, and in alignment with the fluid dispensing member 36 to form a fluid path, is a fluid distributing member, illustrated herein as a needle 40 having a sharp or piercing end 42. While shown as a needle, the fluid distributing member can be any object that has a lumen or interior passageway to allow fluid the flow through. The sharp or piercing end 42 extends within the interior portion formed by the external surface 31 and continuous side wall 35, see FIG. 3A or 3B. However, unlike traditional dispensing pens, i.e. insulin pens, the sharp or piercing end 42 only extends within the interior of the dispensing tip cap 34. The needle does not extend outwardly towards or through the fluid dispensing member 36. The needle is designed to contact or pierce a septum 37 of the vial or cartridge 26. Fluid moves within the needle and through opening 38 of the fluid dispensing member 36. The dispensing pen 10 is secured together by mating female/male threading 43 of the lower elongated body component 20 with the corresponding male/female threading 45 of the upper elongated body component 18, see FIG. 2.

In use, when a user pushes the button 22 positioned at the first end 12, a shaft 46 (see FIG. 2) inside of the elongated body 16 travels a precisely controlled distance. The shaft 46 pushes a plunger or piston 48 within open portion 50 of the vial or cartridge 26, contacting a movable stopper 52. movable stopper 52 may be made of a rubber. The movable stopper prevents the dispensing material or solution 28 from dispensing from the vial towards in the wrong direction. However, when pushed the movable stopper 52 applies a pressure towards the opposing end 14. The force results in pushing the dispensing material or solution 28 from the vial into the needle 40, and out to the individual via the fluid dispensing member 36. As there is no needle in the exterior portion of the dispensing tip cap 34, fluid can be dispensed to various anatomical human orifices (mouth, anal opening, urinary opening, vaginal opening, ear, nose or nasal passage opening, or eye) without the fear of piercing directly thought the skin or other organ. As such, the dispensing pen 10 can be used to self administer or dispense fluid safely and easily into one's body orifice.

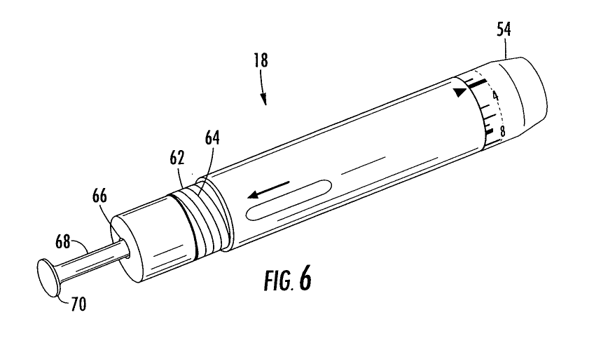

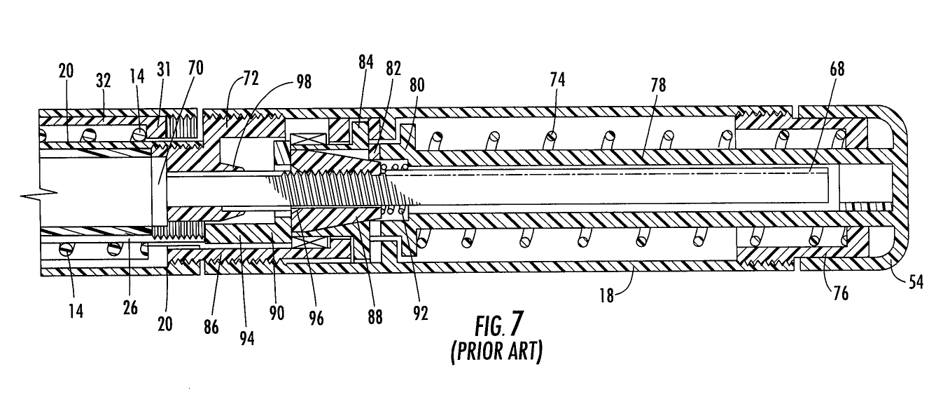

The dispensing pen 10 can be adapted to dispense a material, such as a fluid, solution, gel, or other materials in a metered mechanism. FIGS. 4-8 illustrate the dispensing pen 10 having dose metered capability to dispense the material in a predetermined amount. In this manner, an individual can dispense predetermined doses or amounts, such as 2 ml to 4 ml, each time the device is used. Having the capability of delivering predetermined doses at each use allows the user to more accurately deliver the needed material when required. Moreover, by knowing that a predetermined amount is being dispensed, the user can leave a certain amount of material, say a 30 day supply, within the device, using only the amount required per usage/dosage.

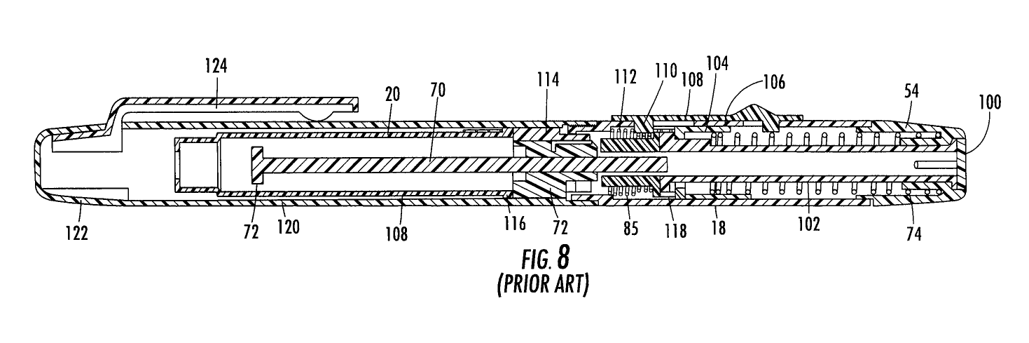

Referring to FIGS. 4 and 5, the dispensing pen 10 is shown having dose metering capability. The dispensing pen 10 has most of the same features as that previously described, with the same features having the same reference number as previously described. The upper elongated body component 18 of the dispensing pen 10 is adapted to provide the mechanism for delivering metered amounts of the material or solution 28. Such metering capability of distributing predetermined amounts can be achieved by using a device have metering components as described in U.S. Pat. No. 5,104,380, (more fully described in FIGS. 7 and 8) the disclosure of which is herein incorporated by reference. Instead of using a push button mechanism to dispense materials all at once, the dispensing pen 10 illustrated in FIGS. 4 and 5 use a mechanism in which the user rotates a dose setting cap 54 concentrically mounted to the upper elongated body component 18. The dose setting cap 54 can be traversed from a first set position to a second selected set position by rotating the dose setting cap 54. Positioned on the dose setting cap 54 are one or more indicating references, illustrated as long or short lines 56 and numbers 58. The long or short lines 56 and numbers 58 indicate the amount of material or solution 28 to be discharged, such as 4 ml or 8 ml. A set selected amount indicator 60 may be included to allow the user visual confirmation as to the actual amount selected. The dosage setting mechanism may further utilize a latch configured to retain the device in the set position and a mechanism to release the latch to allow the dosed material to be discharged.

The upper elongated body component 18 acts as a plunger guide. The elongated body component 18 further comprises a cylindrical boss 62 extending out way from the body and is externally threaded to accommodate the lower elongated body component 20 (screws into the threading 43 of the lower upper elongated body component 20). An opening or bore 66 allows a shaft 68 with plunger 70 to move from end 12 into the vial or cartridge 26. As the shaft 68 with plunger 70 moves forward, it engages the movable stopper 52 placed within the vial or cartridge 26. As the movable stopper 52 moves, it exerts a force on the material or solution 28, pushing it to the dispensing tip cap 34, expelling the fluid out to the individual through the fluid dispensing member 36. The opening or bore 66 may contain opposed flats to preventing rotation of the plunger 70 as it moves into the vial or cartridge 26.

FIG. 7 illustrates a cross sectional view of an embodiment of the internal components which comprise the dose setting dispensing mechanism or assembly. The components which comprise the dose setting dispensing mechanism or assembly shown in FIG. 7 is an illustrative embodiment described in U.S. Pat. No. 5,104,308, and have been included to provide an illustrative embodiment of a dose setting dispensing mechanism or assembly. As shown, the upper elongated body component 18 is closed at one end by plunger guide 72 screwed into the body and having a cylindrical boss extending to the left and being externally threaded to accommodate the lower elongated body 20. The plunger guide 72 guides longitudinal movement of the plunger from a recess in the end of the plunger guide 72, progressively into the open end of the vial or cartridge 26. Movement of the plunger 70 may be accomplished by energy stored in a helical spring 74, which is twisted as a pre-set dose of the material or solution 28 to be dispensed. The amount to be injected can be set by rotation of the dose setting cap 54 which can turn about a graduated sleeve 76 at the right-hand end of the upper elongated body component 18.

The dose setting cap 54 maybe formed integral with a drive sleeve 78. The drive sleeve 78 can turn in a plain bearing defined by the graduated sleeve 76 and has an annular ring 80 projecting at its left-hand end and formed with a ring of ratchet teeth. The helical spring 74 may be secured, respectively, at its ends to a flange at the left-hand end of the drive sleeve 78 and a flange on the sleeve 72 so that, as dose setting cap 54 is turned, the helical spring 74 is strained by being partially wound up. The helical spring 74 may be a compression spring, and when strained torsionally (and unstrained) urges the teeth on the annular ring 80 of the drive sleeve 76 into engagement with cooperating ratchet teeth 82 formed on a ratchet gear 84. The arrangement of the ratchet teeth 82 is such that the dose setting cap 54 and drive sleeve can be turned in relation to the ratchet gear 84 in one direction only, with turning being accompanied by a series of clicks, as each successive ratchet tooth 82 is engaged. The ratchet gear 84 is normally held against rotation by engagement of external teeth on the ratchet gear 84 with internal teeth on a locking cage 86. The locking cage 86 has legs which extend through slots in the plunger guide 72 to prevent rotation of the locking cage 86 in relation to the upper elongated body component 18. The locking cage 86 can slide axially. When the preset or dose of dispensable material or solution 28 is to be injected, the locking cage 86 can be slid axially out of engagement with the ratchet gear 84 so that the gear can rotate, driven by the torque of the helical spring 74 through the drive sleeve 78 and the ratchet teeth 82. Rotation continues until the dose setting cap 54 returns to its initial position defined by a positive stop between the dose setting cap 54 and the upper elongated body 18.

Within the ratchet gear 84 are a pair of opposed drive tapers 88 which are externally splined to be capable of axial sliding in relation to the ratchet gear 84 and are internally threaded with a quick pitch thread to cooperate with a corresponding external thread on the stem of the plunger 70. Rotation of the ratchet gear 84 and drive tapers 88 is accompanied by axial movement of the plunger along the quick pitch thread since the plunger stem cannot turn in the plunger guide 72. Thus, the plunger is driven into the vial or cartridge 26, expelling the preset dose of the dispensable material or solution 28. When the dose setting cap 54 stops turning on the upper elongated body component 18, the plunger 70 remains part-way along the inside of the vial or cartridge 26. The procedure can be repeated until the vial or cartridge 26 is exhausted, after which the vial or cartridge 26 can be replaced by unscrewing the lower elongated body component 20 from the plunger guide 72. A reset ring 90 normally holds the drive tapers 88 in the tapered splines in the ratchet gear 84 against a compression spring 92 so that the quick pitch thread drive is normally engaged. However, when the lower elongated body component 20 has been unscrewed from the plunger guide 72, the reset ring 90 can be slid by the action of the compression spring 92, acting through the drive tapers 88. The reset ring 90 has legs 94 which can slide in the same slots in a plunger guide 72 which accommodate the legs of the locking cage 86. The drive tapers 88 can be pushed by the compression spring 92 until a counter-sink 96 formed in the left-hand face of the two joined drive tapers 88 move over an inclined projection 98 on the right-hand end of the plunger guide 72. Such action opens the drive tapers 88 so that the quick pitch thread connection with the plunger stem is removed and the plunger 70 can be pushed back into the upper elongated body component 18.

FIG. 8 a sectional elevation of the second embodiment described in U.S. Pat. No. 5,104,380. The dose is preset by turning the dose setting cap 54 about the upper elongated body component 18. The dose setting cap 54 may be coupleable with an end cap 100 and the drive sleeve 102 and one end of the helical spring 74. The other end of the helical spring 74 may be locked to a spring retainer 104, which fits inside the upper elongated body component 18 and may be keyed against rotation in relation to the upper elongated body 18 by an external key 106 seated in a notch formed in the body. At the left-hand end of the drive sleeve 102 is an annular flange site within the right-hand end of a drive gear 85 and the flange on the drive sleeve 102 and the drive gear 85 have cooperating ratchet teeth, enabling the drive sleeve to be turned in one direction only in relation to the drive gear 85, which is keyed against rotation in the upper elongated body component 18 by means of a trigger slide 108. The trigger slide 108 has an internal spline 110 engaging with an external spline on the drive gear 85. The drive sleeve 102 may be formed integrally at the left-hand end with a circumferentially extending arm having at its free end a radially outwardly extending ratchet tooth, which is urged by the resilience of the arm into engagement with a ring of ratchet teeth on the drive gear 85. As the dose setting cap 54 is turned, the user can count the clicks as the ratchet tooth moves over successive teeth on the drive gear 85 and can also observe the movement of a scale on the dose setting cap 54 in relation to a pointer (60) on the upper elongated body component 18 so that he can set the preset dose visually and aurally. A spring 112 urges the trigger slide 108 to the right to maintain its engagement with the drive gear 85 and prevents unwinding of the helical spring 74 until the dose has been set.

The user can slide the trigger slide 108 to the left as defined by stops at the end of a slot formed in the upper elongated body component 18 so that an internal flange on the trigger slide 108 moves to the left compressing the spring 112 and moving the spline 110 out of engagement with the drive gear 85. The spline 110 is no longer retained against rotation so that the helical spring 74 can unwind rotating the drive sleeve 102 and the drive gear 85. The plunger 70 is formed with an integral quick pitch screw thread cooperating with a corresponding internal thread in the bore of the drive gear 85 so that rotation of the drive gear 85 is accompanied by axial movement of the plunger 70. Rotation continues until an external projection on the drive sleeve 102 comes against a stop consisting of an internal projection formed in the spring retainer 104 when the preset dose will have been discharged from the vial or cartridge 26 by the plunger 70. The plunger 70 may be prevented from rotating during that movement by two opposed axial flats formed on the plunger 70 and locating in a correspondingly shaped hole in the plunger guide 72, which also acts as a rewind knob. The plunger guide 72 is normally retained against rotation in the upper elongated body component 18 by a locking bar 114 fitted into an axially extending slot in the plunger guide 72 and having an external tooth cooperating with an internal tooth at the left-hand end of the upper elongated body component 18. The locking bar 114 has a radially inwardly extending piece fitted in a notch in the bottom of the slot and acting as a pivot. When the locking bar 114 is unrestrained, the natural position of its left-hand end is radially outwardly of the position shown so that the tooth at the right-hand end is pivoted radially inwardly out of engagement with the corresponding tooth at the left-hand end of the upper elongated body component 18. When a housing connector 116 is fitted over the plunger guide 72 and the left-hand end of the upper elongated body component 18, it deflects the left-hand end of the locking bar 114 radially inwardly in which the tooth at the right-hand end is in engagement with the upper elongated body component 18.

For another dose, the dose setting cap 54 is rotated through the desired number of stops after the trigger slide 108 has been moved by the spring 112 back to the right-hand position, and then when the trigger slide 108 is operated, a further dose is ejected. This continues until the vial or cartridge 26 is exhausted, after which the lower elongated body component 20 is removed from the body by unscrewing the housing connector 116. That releases the plunger guide 72 for rotation in relation to the body so that it acts as a rewind knob and can be turned by hand to drive the plunger 70 back to the right to allow a new vial or cartridge 26 to be inserted. When the housing connector 116 is re-engaged, the rewind knob is locked in position ready for further operation. The right-hand end of the plunger 70 may carry a C clip 118 for defining the extreme left-hand position of the plunger 70. A removable cover 120 with a cap 122 and a pen type clip 124 may be sued and are fitted over the left-hand end when the device is not in use. Turning the dose setting cap 54 through the desired number of clicks in accordance with the preset dose and sliding the slide trigger 108 to the left allows release of the preset dose.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A system for administering a material to an individual comprising:
a dispensing device having a first end, a second end, and an elongated body, said elongated body sized and shaped to hold a vial containing a material to be distributed and stored within said vial, and a mechanism adapted to dispense said material within said vial to a mammal;
a dispensing tip cap secured to one end of said dispensing device.

2. The system for administering a material to an individual according to claim 1 further including a vial containing a material to be dispensed.

3. The system for administering a material to an individual according to claim 1 wherein said dispensing tip cap further comprises exterior surface having a dispensing member projecting outwardly from said external surface.

4. The system for administering a material to an individual according to claim 3 wherein said dispensing tip cap further comprises an needle having a pointed end extending from an interior surface into an interior region, whereby said needle sized and shaped to engage with a vial having a material to be dispensed stored within said elongated body.

5. The system for administering a material to an individual according to claim 1 wherein said elongated body comprises a first, upper elongated body member, said first, upper elongated body member housing a mechanism for dispensing a material to be distributed and stored within said vial.

6. The system for administering a material to an individual according to claim 5 wherein said elongated body further comprises a second, lower elongated body member, said second, lower elongated body member sized and shaped to house vial containing a material to be dispersed.

7. The system for administering a material to an individual according to claim 1 wherein said dispensing device further contains a dose setting mechanism configured to dispense a predetermined amount of material to be dispensed.

8. The system for administering a material to an individual according to claim 7 wherein said dose setting dispensing mechanism further includes a rotatable cap.

9. The system for administering a material to an individual according to claim 5 wherein said dispensing device further contains a dose setting mechanism configured to dispense a predetermined amount of material to be dispensed.

10. The system for administering a material to an individual according to claim 9 wherein said dose setting mechanism further includes a rotatable cap.
